(19) United States
(12) Patent Application Publication
(10) Pub. No.: US 2023/0364349 A1
(43) Pub. Date: Nov. 16, 2023

(54) Dose Setting Assembly for a Medical Injection Device

(71) Applicant: Becton Dickinson France, Le Pont de Claix (FR)

(72) Inventors: Longxiang Huang, Suzhou (CN); Yan Liu, Shanghai (CN)

(21) Appl. No.: 18/247,087

(22) PCT Filed: Sep. 29, 2021
(86) PCT No.: PCT/EP2021/076838

§ 371 Date: Mar. 29, 2023

(30) Foreign Application Priority Data
Sep. 30, 2020 (EP) 20306138.7
Jan. 25, 2021 (CN) 202110099131.X

(51) Int. Cl. A61M 5/315

(52) U.S. Cl. CPC A61M 5/31548

(57) ABSTRACT

The dose setting assembly for a medical injection device including a barrel defining a reservoir adapted to contain a medical injection fluid, an open proximal end and a distal end including a hub portion, a flange disposed at the proximal end of said barrel, and an elongated plunger rod having a plunger flange at a proximal end and a stopper a distal end. The plunger rod being able to slidably move within the barrel, includes a backstop attachable to the flange of the barrel and a dose setting member secured on the backstop, configured to maintain the plunger rod in a predetermined position, the dose setting member being transversally removable from the backstop to authorize the plunger rod to move towards the backstop and abuts onto said backstop.

### CROSS-REFERENCE TO RELATED APPLICATIONS

This application is the United States national phase of International Application No. PCT/EP2021/076838 filed Sep. 29, 2021, and claims priority to European Patent Application No. 20306138.7 filed Sep. 30, 2020, and Chinese Patent Application No. 202110099131.X filed Jan. 25, 2021, the disclosures of which are hereby incorporated by reference in their entireties

### BACKGROUND OF THE INVENTION

### Field of the Invention

The invention relates to a dose setting assembly for a medical injection device such as a syringe which enables to dispense an accurate dose of medical injection fluid.

### Description of Related Art

As it is known in the art, medical injection device such as syringes are utilized to administer a medical injection fluid in a liquid form to a patient.

Syringes typically include a barrel adapted to retain the medical injection fluid. A plunger rod is inserted through the open distal end of the barrel. Through its engagement with an elastomeric or rubber-like stopper fitted in a fluid-tight manner within the interior of the barrel, a user can apply a manual force to the plunger proximal end to deliver the medical injection fluid through a fluid passageway provided at a the distal end of the barrel. A barrel flange is typically provided around the open proximal end of the syringe barrel as a form of finger rest to facilitate a user's manipulation of the medical injection device and a plunger flange is also provided at the proximal end of the plunger rod whereon a user can apply digital pressure. The medical injection fluid is then dispensed by moving the plunger forward through the barrel.

Dispensing an accurate volume of medical injection fluid can be critical like in the case, for example, of an injection of a medicament in an eyeball.

To this end, the barrel is conventionally marked with graduation lines, which determine a volume of medical injection fluid dispensed by a stroke of the plunger. A desired dosage is determined by the distance between two graduation lines.

The graduation lines are generally closely spaced apart and can be difficult to read and thus dispensing an accurate dose of medical injection fluid can be difficult.

Furthermore, the break-away (static friction) required to begin plunger movement is greater than the glide-force (dynamic friction) required once the plunger has begun moving. Thus, it is easy for a user to over-shoot a desired dose when precise movement is required.

Thus, when the user is not being very careful, too little or too much medical injection fluid can be delivered to a patient.

### SUMMARY OF THE INVENTION

In this context, it is desired to improve accuracy of medical injection fluid dispensing from a medical injection device such as a syringe.

This and other related concerns are addressed by a dose setting assembly for a medical injection device including a barrel defining a reservoir adapted to contain a medical injection fluid, an open proximal end and a distal end including a hub portion, a flange disposed at the proximal end of said barrel, and an elongated plunger rod having a plunger flange at a proximal end and a stopper at a distal end, the plunger rod being able to slidably move within the barrel. Said dose setting assembly comprises:
a backstop attachable to the flange of the barrel and
a distal dose setting member removably secured to the backstop configured to maintain the plunger rod in a distal predetermined position, the distal dose setting member being transversally removable from the backstop to authorize the plunger rod to move towards the backstop and abuts onto said backstop, and
a proximal dose setting member secured on the distal dose setting member, maintaining the plunger rod in a second predetermined position, the proximal dose setting member being transversally removable from the distal dose setting member to authorize the plunger rod to move towards the distal dose setting member and abuts onto said distal dose setting member.

The invention provides for an add-on assembly, which can be readily applicable on a conventional medical injection device such as a plastic syringe or a glass syringe. The invention makes it possible to maintain a syringe plunger rod in a pretermitted position by a dose setting member. After removing said dose setting member, the plunger is free to distally advance. The distal motion of the plunger is limited by the plunger flange which abuts onto the backstop. The stroke of the plunger is thus entirely controlled and the volume of medical injection fluid, which is dispensed accordingly, does not rely on a user reading graduations on the barrel. It also ensure that priming the injection device is carried out in a controlled manner.

In an embodiment, the proximal dose setting member includes at least one inner locking means configured to maintain the plunger flange in a concealed locked position. This embodiment of the invention ensures that the plunger rod does not unintentionally move during sterilization, storage and transportation.

Other embodiments of the invention may include the following features taken separately or in combination:
the dose setting member includes an elongated U-shaped wall defining an enclosure.
the dose setting member includes a distal part provided with at least one external groove oriented perpendicularly to the dose setting member longitudinal axis and a proximal part provided with at least one internal groove oriented perpendicularly to the dose setting member longitudinal axis.
the dose setting member includes an internal shoulder provided in the U-shaped wall and an external shoulder provided in the U-shaped wall.
the external grooves of the distal part of a distal dose setting member are dimensioned and configured to fit into the internal grooves of the proximal part of a proximal dose setting member.
the backstop includes a distal plate and a proximal funnel, which both define a longitudinal enclosure.
the backstop includes a distal groove adjacent the distal plate and two arcuate portions provided on the distal plate.
the backstop includes a proximal groove provided in the proximal funnel.
the distal plate extends radially from the funnel.

Another aspect of the invention concerns a method of operating a medical injection device, comprising the steps of:
providing a medical injection device having a dose setting assembly;
removing the at least one dose setting member from the medical injection device.

In one embodiment, the dose setting member is the proximal dose setting member. The proximal dose setting member allows protection of the plunger rod since it is entirely concealed within the proximal dose setting member. Thus, the plunger rod cannot move preventing external damages, allowing transportation or storage in a sterile zone, and preventing accidental or inadvertent expel of the medical product contained in the barrel of the syringe.

The removal of the proximal dose setting member allows the user to have access to the plunger rod of the syringe. The removal of the proximal dose setting member is carried out off a living human or animal body. The user may then advance the plunger rod in a distal direction by applying digital pressure to the plunger flange until the plunger flange contacts a proximal area of the distal dose setting member. This enables priming the syringe. In other words, the expulsion of air bubbles is performed while defining precisely the medical dose by expulsing the non-desired quantity of medical dose and air bubbles.

In one embodiment, the dose setting member is the distal dose setting member.

The removal of the distal dose setting member permits freeing the plunger rod which was previously abutting on a proximal area of the distal dose setting member. The removal of the distal dose setting member is carried out off a living human or animal body.

In one embodiment, the method further comprises the step of moving the plunger rod into the syringe barrel towards the backstop until the barrel flange abuts against the backstop.

In this embodiment, the user may advance the plunger rod in the distal direction by applying digital pressure to the plunger flange until the plunger flange contacts a proximal area of the backstop. Thus, a predetermined dose of medical injection fluid may be expelled from the reservoir allowing the delivery of a precise therapeutic dose. In this step, the precise therapeutic dose may be carried out on a living human or animal body.

In another embodiment, the method of operating can further include the steps of:
providing a medical device having a dose setting assembly;
removing the proximal dose setting member from the distal dose setting member;
moving the plunger rod into the syringe barrel towards the distal dose setting member until the plunger rod abuts onto the distal dose setting member;
removing the distal dose setting member from the backstop.

In one embodiment, the method further comprises the step of moving the plunger rod into the syringe barrel towards the backstop until the plunger rod abuts onto the backstop.

In an embodiment of the invention, the dose setting member is transversely removed from the medical injection device.

In an embodiment of the invention, the method is not carried out on a living human or animal body.

The disclosure set forth herein is also defined by the following features:

A dose setting assembly for a medical injection device including a barrel defining a reservoir adapted to contain a medical injection fluid, an open proximal end and a distal end including a hub portion, a flange disposed at the proximal end of said barrel, and an elongated plunger rod having a plunger flange at a proximal end and a stopper a distal end, the plunger rod being able to slidably move within the barrel. Said dose setting assembly comprises:
a backstop attachable to the flange of the barrel and
a dose setting member removably secured on the backstop, configured to maintain the plunger rod in a predetermined position, the dose setting member being transversally removable from the backstop to authorize the plunger rod to move towards the backstop and abuts onto said backstop.

The invention provides for an add-on assembly, which can be readily applicable on a conventional medical injection device such as a plastic syringe or a glass syringe. The invention makes it possible to maintain a syringe plunger rod in a pretermitted position by a dose setting member. After removing said dose setting member, the plunger is free to distally advance. The distal motion of the plunger is limited by the plunger flange which abuts onto the backstop. The stroke of the plunger is thus entirely controlled and the volume of medical injection fluid, which is dispensed accordingly, does not rely on a user reading graduations on the barrel.

In an embodiment of the invention, the dose setting assembly includes a distal dose setting member secured on the backstop configured to maintain the plunger rod in a distal predetermined position the distal dose setting member being transversally removable from the backstop to authorize the plunger rod to move towards the backstop and abuts onto said backstop, and
a proximal dose setting member secured on the distal dose setting member, maintaining the plunger rod in a second predetermined position, the proximal dose setting member being transversally removable from the distal dose setting member to authorize the plunger rod to move towards the distal dose setting member and abuts onto said distal dose setting member. In this embodiment, the invention makes it possible to precisely define a dose of medical injection fluid. It also ensure that priming the injection device is carried out in a controlled manner.

In an embodiment, the proximal dose setting member includes at least one inner locking means configured to maintain the plunger flange in a concealed locked position. This embodiment of the invention ensures that the plunger rod does not unintentionally move during sterilization, storage and transportation.

Other embodiments of the invention may include the following features taken separately or in combination:
the dose setting member includes an elongated U-shaped wall defining an enclosure.
the dose setting member includes a distal part provided with at least one external groove oriented perpendicularly to the dose setting member longitudinal axis and a proximal part provided with at least one internal groove oriented perpendicularly to the dose setting member longitudinal axis.
the dose setting member includes an internal shoulder provided in the U-shaped wall and an external shoulder provided in the U-shaped wall.
the external grooves of the distal part of a distal dose setting member are dimensioned and configured to fit into the internal grooves of the proximal part of a proximal dose setting member.
the backstop includes a distal plate and a proximal funnel, which both define a longitudinal enclosure.
the backstop includes a distal groove adjacent the distal plate and two arcuate portions provided on the distal plate.
the backstop includes a proximal groove provided in the proximal funnel.
the distal plate extends radially from the funnel.

Another aspect of the invention concerns a method of operating a medical injection device, comprising the steps of:
providing a medical injection device having a dose setting assembly;
removing the at least one dose setting member from the medical injection device;
moving the plunger rod into the syringe barrel towards the backstop until the barrel flange abuts against the backstop,

In another embodiment, the method of operating can further include the steps of:
providing a medical device having a dose setting assembly;
removing the proximal dose setting member from the distal dose setting member;
moving the plunger rod into the syringe barrel towards the distal dose setting member until the plunger rod abuts onto the distal dose setting member;
removing the distal dose setting member from the backstop;
moving the plunger rod into the syringe barrel towards the backstop until the plunger rod abuts onto the backstop.

In an embodiment of the invention, the dose setting member is transversely removed from the medical injection device.

Further advantages and advantageous features of the invention are disclosed in the following description and in the dependent claims.

### BRIEF DESCRIPTION OF THE DRAWINGS

The invention will now be described in greater detail by way of reference to the appended drawings, wherein:

FIG. 1 depicts in perspective view, a dose setting assembly according to an exemplary embodiment of the present invention mounted to a conventional syringe;

FIG. 2 is a side view of the embodiment depicted in FIG. 1;

FIG. 3 depicts in perspective view a backstop of the dose setting assembly of FIG. 1;

FIG. 4 depicts in perspective view a dose-setting member of the dose setting assembly of FIG. 1;

FIGS. 5 to 7 illustrate the dose setting assembly of FIG. 1 in operation;

FIG. 8 depicts in perspective view, a dose setting assembly according to an exemplary embodiment of the present invention mounted to a conventional syringe;

FIG. 9 depicts, on an enlarged scale, the dose setting assembly of FIG. 8;

FIGS. 10 to 13 illustrate the dose setting assembly of FIG. 8 in operation;

FIG. 14 depicts, on an enlarged scale, a distal setting member which is included in the dose setting assembly of FIG. 8.

### DESCRIPTION OF THE INVENTION

As used herein, the term “proximal” and derivatives thereof, shall mean the end of an item or direction closest to the user during use of the subject invention. The term “distal” and derivatives thereof, shall mean the end of an item or direction towards a patient during use of the subject invention. As used herein, the term “medical injection fluid” and derivatives thereof shall mean any substance that is intended for injection into a patient, including, by way of non-limiting example, drugs, vaccines, therapeutics, and the like. It will be obvious to a person of skill in the art, and from the disclosure provided herein, that the subject invention is not limited or otherwise defined by the type or class of substance administered using the inventive dose setting assembly.

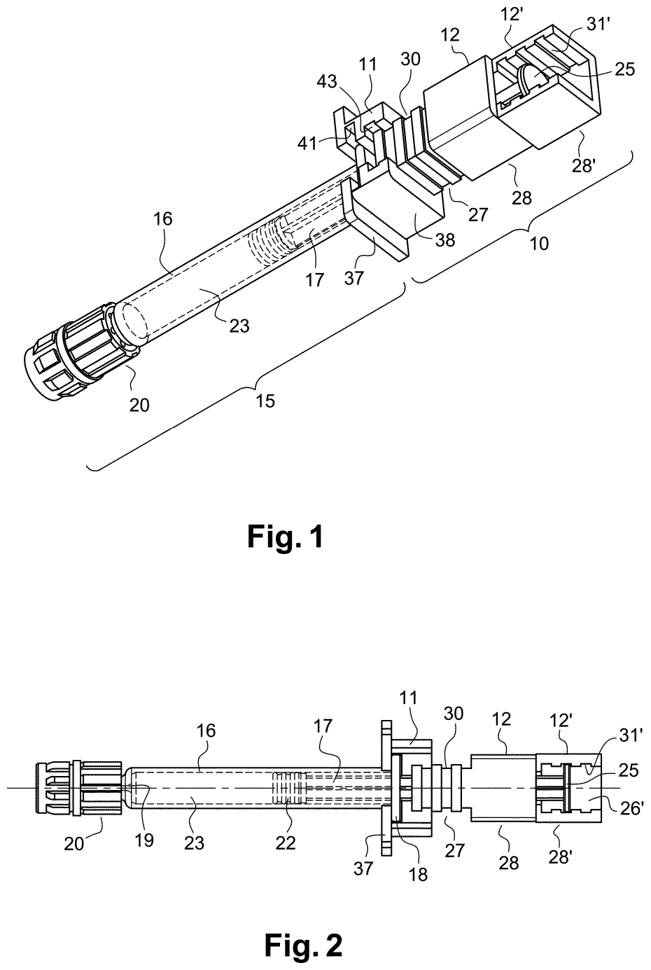

In an exemplary embodiment of the present invention, as shown in FIGS. 1 to 7, a dose setting assembly 10 includes a backstop 11, a distal dose setting member 12 and a proximal dose setting member 12′.

The dose setting assembly 10 engages a conventional syringe 15 that includes a barrel 16 and an elongated plunger rod 17.

The barrel 16 includes an open proximal end having a flange 18 and a distal end including a tip having a passageway 19.

The distal end of the barrel 16 can be attached to a connecting hub 20. In other embodiments, a needle assembly can be attached to the distal end of the barrel 16.

The plunger rod 17 is inserted into the barrel 16 at the proximal end of the syringe 15. At the distal end of the plunger rod 17 is a stopper 22, which is used to seal the barrel 16 to form a reservoir 23 for a medical injection fluid. The plunger rod 17 also includes a plunger rod flange 25 at the proximal end of the plunger rod 17. The plunger rod flange 25 extends radially from the plunger rod 17. In the illustrated embodiment, the plunger rod flange 25 has the shape of disk.

At the proximal end of the syringe 15 is the dose setting assembly 10.

Turning now more particularly to FIG. 3, one embodiment of the dose setting member 12 features an elongated U shaped single wall 24 defining an enclosure 26; the enclosure 26 includes a longitudinal opening 29 defined by the free ends of the U-shaped wall 24. In the illustrated embodiment, the dose-setting member 12 has a square section. The dose-setting member 12 could also have a substantially circular section, or any other shaped-section.

The dose-setting member 12 includes a distal part 27 dimensioned and configured to connect the backstop 11 and a proximal part 28 dimensioned and configured to accommodate a proximal dose-setting member 12′ as will be explained in more detail hereinafter.

The distal part 27 features a series of external grooves 30 formed on the external surface of the U shape wall 24 and the proximal part features a series of internal grooves 31 formed on the internal surface of the U shaped wall 24. External grooves 30 and internal grooves 31 are parallel and are oriented perpendicularly to a dose setting member longitudinal axis. The dose-setting member 12 includes a section restriction, which makes the distal part 27 narrower than the proximal part 28. The section restriction includes an internal shoulder 33 provided in the U-shaped wall 24 and an external shoulder 34 provided in the U-shaped wall 24.

The internal grooves 31 are configured and dimensioned to receive the plunger rod flange 25 as will be explained in more details hereafter.

To permit the insertion of a distal dose setting member 12 distal part 27 into a second setting dose member 12′, the external grooves 30 of the distal part 27 are dimensioned and configured to fit into the internal grooves 31 of the proximal part 28.

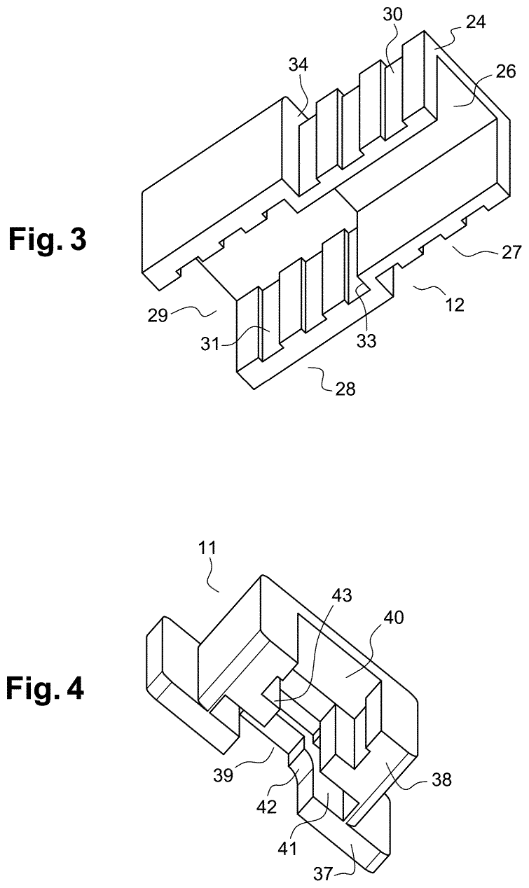

Turning now more particularly to FIG. 4, one embodiment of the backstop 11 features a distal plate 37 and a proximal funnel 38, which both define a longitudinal enclosure 39. The distal plate 37 extends radially from the funnel 38. The enclosure 39 includes a longitudinal opening 40.

To permit the insertion of the backstop 11 onto a syringe 15 as will be explained in more detail hereinafter, the backstop 11 internal wall is provided with a distal groove 41 which is adjacent the distal plate 37. The distal plate 37 further includes two arcuate portions 42.

To permit the insertion of the distal part of the backstop 11 as will be explained in more detail hereinafter, the backstop 11 includes a proximal groove 43 provided in the internal surface of the funnel 38.

It will be appreciated and understood by those skilled in the art that the backstop 11 and the dose-setting member 12 may be formed in any one of conventional manners such as injection molding. It may be formed from appropriate medical grade plastics, hard rubber materials, glass, metals or the like.

Turning now more particularly to FIG. 1 or 2, a dose-setting assembly 10 in accordance with the present invention is configured for mating with a syringe 15 via the syringe flange element 18. It will be appreciated and understood by those skilled in the art, however, that the dose setting assembly 10 in accordance with the invention may be applied to other medical delivery devices, where it is desired to deliver an accurate and repeatable dose of medical injection fluid.

Syringes can be marketed as in a prefilled form wherein a set dosage of a medical injection fluid is provided in the reservoir. In this configuration, the plunger extends from the barrel 16.

As seen in FIG. 2, the dose setting assembly 10 includes a backstop 11, which engages onto the barrel flange 18. The barrel flange 18 engages into the distal groove 41 and the barrel 16 rests on the arcuate portions 42 provided in the backstop 11. The backstop 11 longitudinal opening 29 opening to the enclosure 26 forms a passageways allowing fitting the backstop 11 on the syringe flange 18 with the plunger rod 17 extending from the barrel 16.

A distal dose-setting member 12 engages into the backstop 11. To this end, the distal part of the dose-setting member 12 engages into the groove 43 provided in the backstop funnel 38.

While in FIG. 2, the backstop 11 and the distal dose-setting member 12 face opposite directions, it is appreciated that the distal dose-setting member 12 can be oriented differently with regards to the backstop 11 thanks to its square section.

A proximal dose-setting member 12′ engages into the distal dose-setting member 12. To the end, the distal part 27′ of the second setting member 12′ engages into the proximal part 28 of the distal dose-setting member 12.

It is appreciated that the distal dose-setting member 12 and the proximal dose-setting member 12′ are identical in the illustrated embodiment.

The backstop 11 and the two dose setting members 12,12′ are attached to each other and to the barrel flange 18 by friction fit. Thus, they can be secured or removed from a syringe 15 by hands without any tools or equipment. Therefore operating the dose setting assembly 10 proves to be easy and intuitive.

Longitudinal openings 40 and 29 provided in the backstop 11 and both dose-setting elements 12,12′ permit the placement of the backstop 11 and both dose-setting elements 12,12′ around the plunger rod 17 which extends from the barrel 16. As the backstop 11 and both dose-setting elements 12,12′ approach the syringe 15, the syringe plunger rod 17 is accommodated within the enclosure of the backstop 11 and both dose-setting elements 12,12′.

As depicted in FIG. 2, the plunger rod 17 is secured in its extended position as the plunger flange 25 is encased in the internal grooves 31′ of the proximal dose-setting member 12′. In the illustrated embodiment, the proximal dose-setting member 12′ includes three internal grooves 31′ thus providing three positions where the plunger flange 12 can be secured.

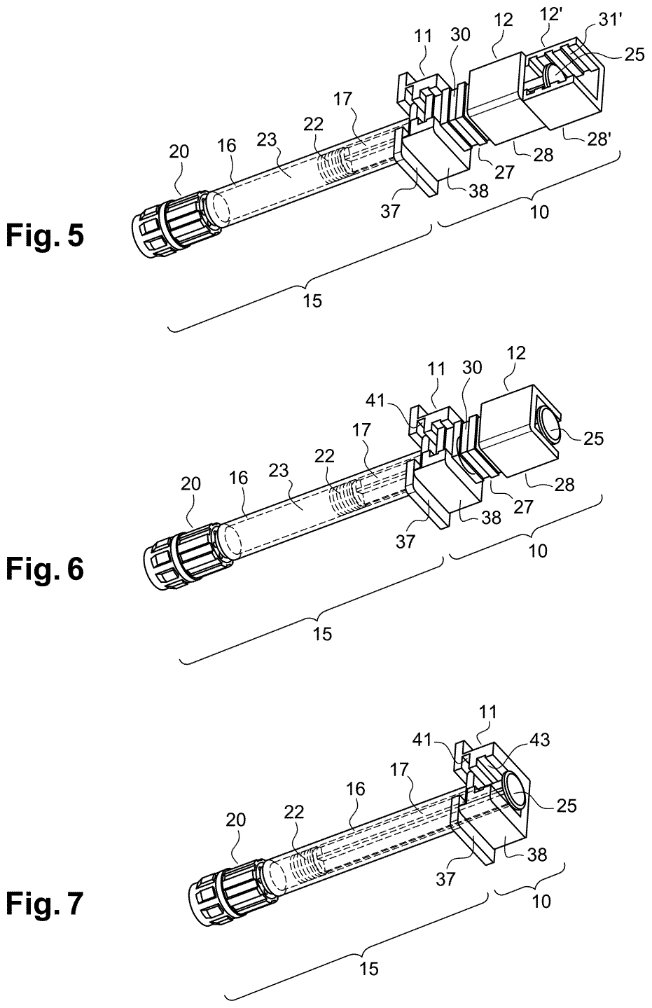

Turning now more particularly to FIGS. 5 to 7, the dose setting assembly 10 is structured to control a syringe standard operations and thus provides a first a medical injection fluid delivery stroke of the plunger allowing accurate priming and a second medical injection fluid delivery stroke of the plunger allowing dispensing of an accurate dose of a medical injection fluid.

The syringe 15 in its configuration of FIG. 3 can be prefilled and can be placed in a sterile packaging (not shown) with the dose setting assembly 10.

After removing the packaging, the syringe 15 is ready to be used. One can note that the plunger rod 17 is entirely concealed within the proximal dose setting member 12′, thus accidental or inadvertent movement of the plunger rod 17 is avoided. Thus, the moving of the plunger stopper outward a sterile zone during the transportation or storage of the syringe 15 is avoided. Besides, accidental or inadvertent expel of the medical product contained in the barrel 16 of the syringe 15 is avoided.

The proximal dose setting member 12′ can be removed from the injection device 15. To this end, the proximal dose setting member 12′ is pulled transversally, this has the effect of making accessible the plunger rod 17 which was previously concealed within the dose setting member 12′.

It can be appreciated that the backstop 11 distal plate 37 forms a type of finger rest permitting the user to easily manipulate the syringe 15 during use.

The user now advances the plunger rod 17 in the distal direction by applying digital pressure to the plunger flange 25. The distal motion of the plunger rod 17 is limited by the plunger flange 25 contacting a proximal area of the distal dose-setting member 12. A dose of medical injection fluid is thus expelled from the reservoir 23 under the plunger motion. This enables priming the syringe 15. In other words, the dose setting assembly makes it possible to dispense the required amount of medical injection fluid to prime the syringe 15 without over dispensing the medical injection fluid. This makes it possible to exhaust the potential air bubbles present in the medical injection fluid thus avoiding injecting them in the patient.

The distal dose-setting member 12 can now be removed from the backstop 11. To this end, the distal dose setting member 12 is pulled transversally, this has the effect of freeing the plunger rod 17 which was previously abutting on a proximal area of the distal dose setting member 12.

The user can now advance the plunger rod 17 in the distal direction by applying digital pressure to the plunger flange 25. The distal motion of the plunger rod 17 is limited by the plunger flange 25 contacting a proximal area of the backstop 11. A predetermined dose of medical injection fluid is thus expelled from the reservoir 23 under the plunger rod motion. Preferably, this second dose is dimensioned to deliver a precise therapeutic dose.

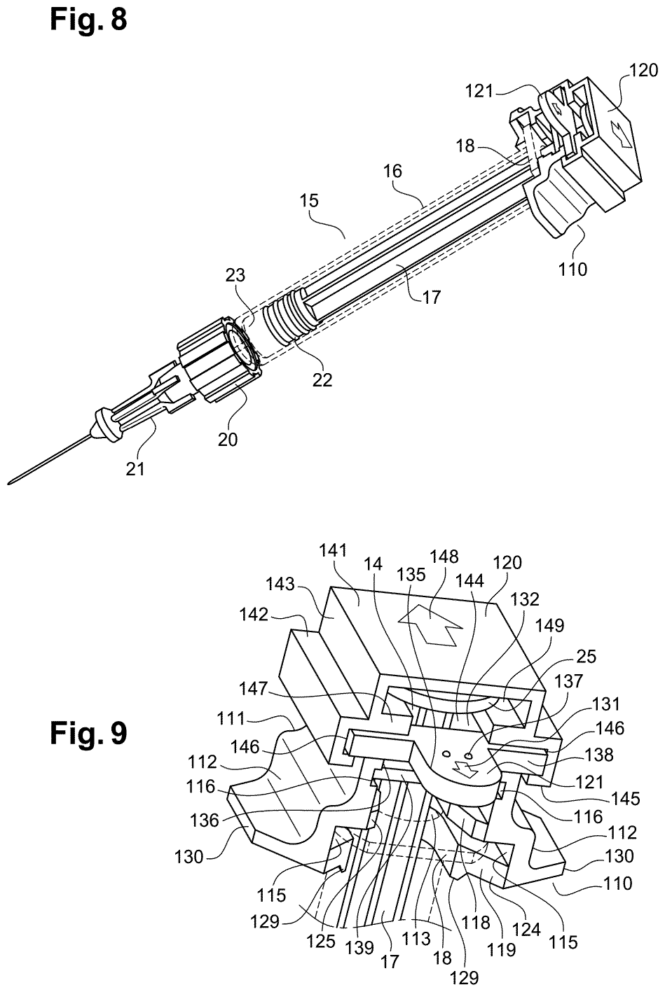

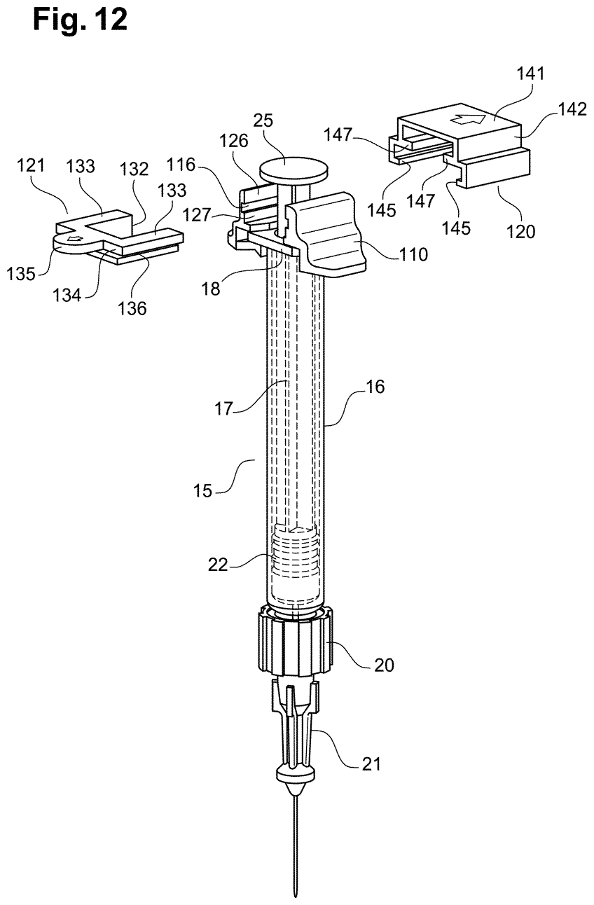

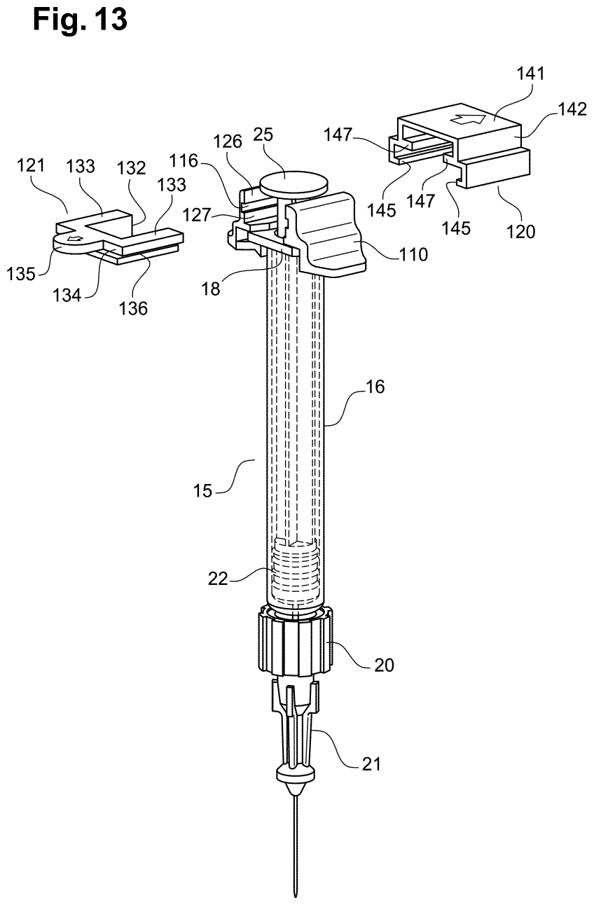

Turning now to FIGS. 8 to 14, one embodiment of the invention includes a static backstop 110 and two removable dose-setting members 120, 121; the two removable dose-setting members 120, 121 define two controlled plunger rod strokes which makes it possible to deliver two accurate doses of medical fluid.

One embodiment of the backstop 110 features a U shaped add-on element which includes a retaining wall linked to two side walls

The backstop 110 includes a transversal wall 111 and two lateral walls 112. An aperture 113 is defined between the transversal wall 111 and the lateral wall 112; the aperture 113 is dimensioned to accommodate passage of syringe plunger 17. The width of aperture 113 is configured greater than the diameter of the plunger rod 17 and is configured smaller than the diameter of the plunger flange 25 as will be explained in more detail hereinafter.

The internal surface of the backstop 110 can be provided with a distal insertion grooves 115 configured to removably connect the backstop 110 on the syringe flange 18 and with an insertion proximal groove 116 configured to removably connect the distal dose-setting member 121 to the backstop 110.

The distal groove 115 is formed between two parallel superimposed flanges 118,119 which protrude inwardly. The superimposed flanges 118,119 can include slanting insertion edges 124,125.

The proximal groove 116 is formed between two parallel flanges 127,126 which protrudes inwardly.

To enhance locking retention of the backstop 110 onto the syringe barrel 16, the backstop 110 can suitably include two lips 129 which extend distally from the flange 119.

The backstop 110 includes an external stepped configuration which provides a finger rest surface 130.

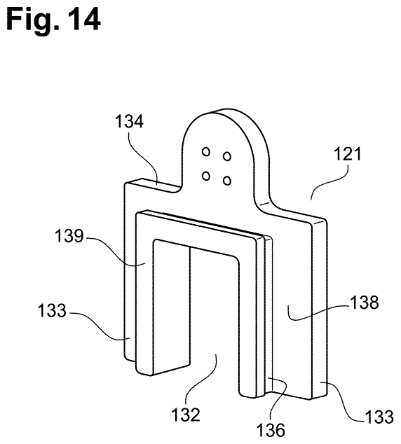

Turning more particularly to FIG. 14, the distal dose-setting member 121 features a general fork shaped configuration which defines an aperture 132. The proximal dose-setting member 121 includes two parallel legs 133 linked by a transversal portion 134. The aperture 132 is defined between the parallel legs 133 and the transversal portion 134; the aperture 132 is dimensioned to accommodate passage of the syringe plunger 16 and is dimensioned so that legs 133 and transversal portion 134 serve as a resting surface for the plunger flange 25. To this end, the width of the aperture 132 is configured greater than the diameter of the plunger rod 17 and is configured smaller than the diameter of the plunger flange 25.

Each leg 133 is provided with an external insertion groove 136. The grooves 136 are defined between a proximal flange 138 and a distal flange 139 which protrude outwardly.

The distal flange 139 is configured and dimensioned to removably engage the proximal groove 116 provided in the backstop 110.

The proximal flange 138 is configured and dimensioned to protrude laterally outward the backstop lateral wall 112 when the distal dose-setting member 121 is engaged on the backstop 110.

The transversal portion 134 is provided with a pulling tab 135 which extends in a direction opposite the legs 133 direction. Tab 135 can be suitably provided with an arrow sign 131 outwardly oriented and may include at least one projection 137 to allow easy grasping of said tab 135.

The proximal dose setting member 120 is configured to be removably connected onto the backstop 110.

With continued reference to FIGS. 8 to 13, the proximal dose setting member 120 features general U shaped configuration including a substantially flat proximal wall 141 and two lateral stepped walls 143 which include a shoulder 142. As best seen on FIG. 9, the proximal wall can feature an arrow sign 148.

Proximal dose setting member 120 includes two inwardly projecting middle ribs 147. The two middle ribs 147 face each other and define an aperture 144 which is dimensioned to accommodate passage of syringe plunger 17 and are dimensioned to serve as a resting surface for the plunger flange 18.

The inner dimension between the middle ribs 147 and the proximal wall 141 may be sized to accommodate the plunger flange 18 so that plunger flange 18 is locked within a locking cavity 149 formed between the proximal wall 141 and the middle ribs 147. In other words, once engaged between the middle ribs 147 and the proximal ribs 134, the plunger flange 18 is locked and thus the plunger rod 17 is unable to move.

Proximal dose setting member 120 may further include two inwardly oriented distal ribs 145. The groove 146 defined between the middle ribs 147 and the distal ribs 145 is dimensioned and configured to removably receive the proximal flange 138 of the distal dose-setting member 121.

It will be appreciated and understood by those skilled in the art that the backstop 110, the proximal dose setting member 120 and the distal dose setting member 121 may be formed in any one of conventional manners such as injection molding. It may be formed from appropriate medical grade plastics, hard rubber materials, glass, metals or the like.

Turning now more particularly to FIG. 8 or 9, a dose-setting assembly 10 in accordance with the present invention is configured for mating with a syringe 15 via the syringe flange element 18. It will be appreciated and understood by those skilled in the art, however, that the dose setting assembly 10 in accordance with the invention may be applied to other medical delivery devices, where it is desired to deliver an accurate and repeatable dose of medical injection fluid.

Syringes can be marketed as in a prefilled form wherein a set dosage of a medical injection fluid is provided in the reservoir. In this configuration, the plunger rod 17 extends from the barrel 16.

As seen in FIG. 8, the backstop 110 engages onto the barrel flange 18. The barrel flange 18 engages into the backstop distal groove 115.

The distal dose setting member 121 in turn connects onto the backstop 110; to this end, the distal flange 139 of the distal setting member 121 engages the proximal groove 116 of the backstop 110.

The proximal dose setting member 120 in turn connects onto the distal dose setting member 121; to this end, the groove 146 of the proximal dose setting member 120 engages the proximal flange 138 of the distal dose setting member 121.

The backstop 110, the proximal dose setting member 121 and the distal dose setting member 121 are attached to each other and to the barrel flange 18 by friction fit. Thus, they can be secured or removed from a syringe 15 by hands without any tools or equipment. Therefore operating the dose setting assembly 100 proves to be easy and intuitive.

In the configuration of the invention illustrated on FIG. 8, the plunger flange 18 is encapsulated within the space defined between the proximal dose setting member 120 and the distal dose setting member 121; this guarantees that the plunger rod 17 cannot be accidently pushed or pulled.

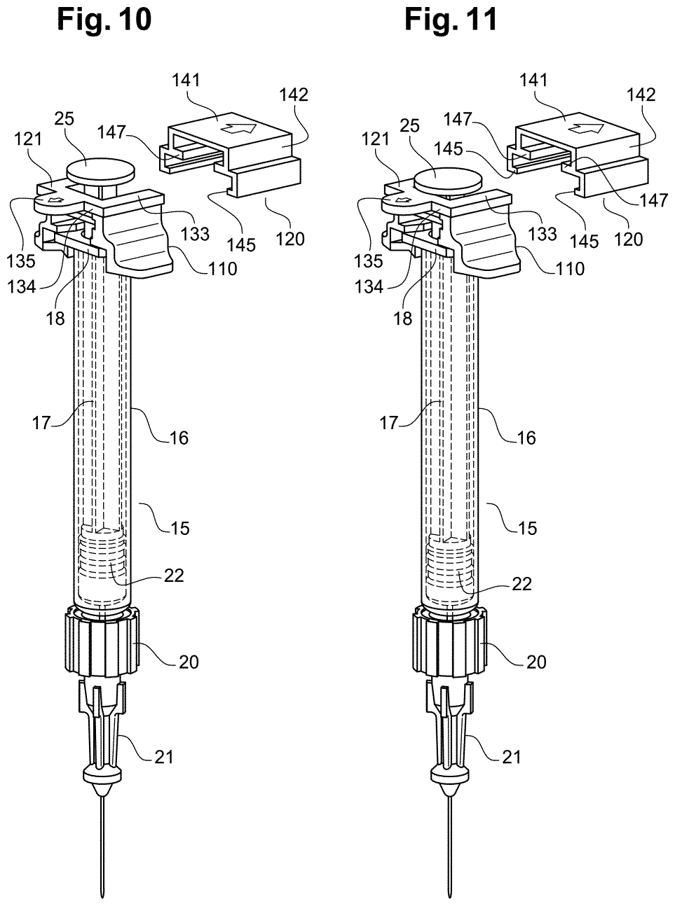

Turning now more particularly to FIGS. 10 to 13, the dose setting assembly 100 is structured to control a syringe standard operations and thus provides a first a medical injection fluid delivery stroke of the plunger allowing accurate priming and a second medical injection fluid delivery stroke of the plunger allowing dispensing of an accurate dose of a medical injection fluid.

The syringe 15 in its configuration of FIG. 8 can be prefilled and can be placed in a sterile packaging (not shown) with the dose setting assembly 100.

After removing the packaging, the syringe 15 is ready to be used. One can note that the plunger flange 18 is encapsulated within the space defined between the proximal dose setting member 120 and the distal dose setting member 121; thus accidental or inadvertent movement of the plunger rod 17 is avoided. Thus, the moving of the plunger stopper outward a sterile zone during the transportation or storage of the syringe 15 is avoided. Besides, accidental or inadvertent expel of the medical product contained in the barrel 16 of the syringe 15 is avoided.

The proximal dose setting member 120 can be removed from the syringe 15. To this end, the proximal dose setting member 120 is pulled transversally following the direction of the arrow sign 148; this has the effect of making accessible the plunger flange 18 which was previously concealed within the proximal dose setting member 120.

The user now advances the plunger rod 17 in the distal direction by applying digital pressure to the plunger flange 25. The distal motion of the plunger rod 17 is limited by the plunger flange 25 contacting the distal dose setting member 121. More precisely the plunger flange 25 abuts the legs 133. A dose of medical injection fluid is thus expelled from the reservoir 23 under the plunger motion. This enables priming the syringe 15. In other words, the dose setting assembly 100 makes it possible to dispense the required amount of medical injection fluid to prime the syringe 15 without over dispensing the medical injection fluid. This makes it possible to exhaust the potential air bubbles present in the medical injection fluid thus avoiding injecting them in the patient.

The distal dose-setting member 121 can now be removed from the backstop 110. To this end, the user can seize the tab 135 so that the distal dose setting member 121 is pulled transversally following the direction of the arrow sign 131. This has the effect of freeing the plunger flange 25 which was previously abutting on the legs 133 of the distal dose setting element 121.

The user can now advance the plunger rod 17 in the distal direction by applying digital pressure to the plunger flange 25. The distal motion of the plunger rod 17 is limited by the plunger flange 25 contacting the proximal flange 126 of the backstop 110. A predetermined dose of medical injection fluid is thus expelled from the reservoir 23 under the plunger rod motion. Preferably, this second dose is dimensioned to deliver a precise therapeutic dose.

It is to be understood that the present invention is not limited to the embodiments described above and illustrated in the drawings; rather, the skilled person will recognize that many changes and modifications may be made within the scope of the appended claims.

1. A dose setting assembly for a medical injection device comprising a barrel defining a reservoir adapted to contain a medical injection fluid, an open proximal end and a distal end comprising a hub portion, a flange disposed at the proximal end of said barrel, and an elongated plunger rod having a plunger flange at a proximal end and a stopper at a distal end, the plunger rod being able to slidably move within the barrel, said dose setting assembly comprising:
a backstop attachable to the flange of the barrel and
a distal dose setting member removably secured to the backstop configured to maintain the plunger rod in a distal predetermined position, the distal dose setting member being transversally removable from the backstop to authorize the plunger rod to move towards the backstop and abuts onto said backstop, and
a proximal dose setting member secured on the distal dose setting member, maintaining the plunger rod in a second predetermined position, the proximal dose setting member being transversally removable from the distal dose setting member to authorize the plunger rod to move towards the distal dose setting member and abuts onto said distal dose setting member.

2. The dose setting assembly of claim 1, wherein the proximal dose setting member comprises at least one inner locking means configured to maintain the plunger flange in a concealed locked position.

3. The dose setting assembly of claim 1, wherein the proximal dose setting member comprises an elongated U-shaped wall defining an enclosure.

4. The dose setting assembly of claim 1, wherein the dose setting member comprises a distal part provided with at least one external groove oriented perpendicularly to the dose setting member longitudinal axis and a proximal part provided with at least one internal groove oriented perpendicularly to the dose setting member longitudinal axis, said at least one internal groove of a proximal dose setting member being configured and dimensioned to receive the plunger flange in a concealed locked position and said at least one external groove being configured and dimensioned to removably engage the at least one internal groove of the distal dose setting member; and said at least one external groove of the distal dose setting member being configured and dimensioned to removably engage the backstop.

5. The dose setting assembly of claim 1, wherein the dose setting member comprises an internal shoulder provided in the U-shaped wall and an external shoulder provided in in the U-shaped wall.

6. The dose setting assembly of claim 4, wherein the external grooves of the distal part of a distal dose setting member are dimensioned and configured to fit into the internal grooves of the proximal part of a proximal dose setting member.

7. The dose setting assembly of claim 1, wherein the backstop comprises a distal plate and a proximal funnel, which both define a longitudinal enclosure.

8. The dose setting assembly of claim 7, wherein the backstop comprises a distal groove adjacent the distal plate and two arcuate portions provided on the distal plate.

9. The dose setting assembly of claim 7, wherein the backstop comprises a proximal groove provided in the proximal funnel.

10. The dose setting assembly of claim 7, wherein the distal plate extends radially from the funnel.

11. A method of operating a medical injection device, comprising the steps of:
providing a medical injection device having a dose setting assembly of claim 1, and
removing the at least one dose setting member from the backstop.

12. A method of claim 11, further comprising the step of:
moving the plunger rod into the syringe barrel towards the backstop until the barrel flange abuts against the backstop.

13. A method of operating a medical injection device, comprising the steps of:
providing a medical device having a dose setting assembly of claim 1;
removing the proximal dose setting member from the distal dose setting member;
moving the plunger rod into the syringe barrel towards the distal dose setting member until the plunger rod abuts onto the distal dose setting member and
removing the distal dose setting member from the backstop.

14. A method of claim 13, further comprising the step of:
moving the plunger rod into the syringe barrel towards the backstop until the plunger rod abuts onto the backstop.

15. The method of claim 11, wherein the dose setting member is transversely removed from the medical injection device.
